(19) United States
(12) Patent Application Publication
(10) Pub. No.: US 2023/0190202 A1
(43) Pub. Date: Jun. 22, 2023

(54) ELECTROPHYSIOLOGICAL SIGNAL MEASUREMENT SYSTEM, ELECTROPHYSIOLOGICAL SIGNAL ADJUSTMENT METHOD AND ELECTRODE ASSEMBLY

(71) Applicant: INDUSTRIAL TECHNOLOGY RESEARCH INSTITUTE, Hsinchu (TW)

(72) Inventors: Heng-Yin CHEN, Zhubei City (TW); Yun-Yi HUANG, Pingtung City (TW); Min-Hsuan LEE, Ji'an Township (TW); Yi-Cheng LU, Hsinchu City (TW); Yu-Chiao TSAI, Tainan City (TW); Bor-Shyh LIN, Tainan City (TW)

(73) Assignee: INDUSTRIAL TECHNOLOGY RESEARCH INSTITUTE, Hsinchu (TW)

(21) Appl. No.: 17/696,065

(22) Filed: Mar. 16, 2022

(30) Foreign Application Priority Data
Dec. 21, 2021 (TW) 110147909

(51) Int. Cl. A61B 5/00; A61B 5/296; A61B 5/28

(52) U.S. Cl. CPC A61B 5/7225; A61B 5/296; A61B 5/28; A61B 2560/0228

(57) ABSTRACT

An electrophysiological signal measurement system, an electrophysiological signal adjustment method and an electrode assembly are provided. The electrophysiological signal measurement system includes an electrode assembly, a variation adjustment device and a signal processing device. The electrode assembly receives an electrophysiological signal, a first electrical characteristic value and a second electrical characteristic value. The variation adjustment device includes a comparison unit and a searching unit. The comparison unit receives the first electrical characteristic value and the second electrical characteristic value, and determines whether a difference between the first electrical characteristic value and the second electrical characteristic value is greater than a threshold. When the difference between the first electrical characteristic value and the second electrical characteristic value is greater than the threshold value, the searching unit searches for several amplitude calibration ratios corresponding to several frequencies. The signal processing device calibrates the electrophysiological signal according to the amplitude calibration ratios.

This application claims the benefit of Taiwan application Serial No. 110147909, filed Dec. 21, 2021, the disclosure of which is incorporated by reference herein in its entirety.

### TECHNICAL FIELD

The disclosure relates in general to an electrophysiological signal measurement system, an electrophysiological signal adjustment method and an electrode assembly.

### BACKGROUND

As people pay more and more attention to the requirements of health management, various kinds of physiological signal measuring devices are constantly being introduced in sports, fitness, health care, nursing, long-term care and other fields.

However, when the coupled electrophysiological signal measuring device is used, the correctness of the signal is often affected by the use of low pressure. Especially when the interface impedance between the skin and the electrode sheet is greater, the baseline of the Electromyography signal (EMG signal) will become more unstable.

In addition, for a subject with muscle injury, the amplitude of the EMG signal will become weak, and it is difficult to measure and interpret.

Furthermore, when the electrophysiological signal is detected at moving state, noise is likely to be generated, and these noises make the interpretation of the electrophysiological signal difficult.

Therefore, an electrophysiological signal adjustment method is required, in order to be able to adjust the electrophysiological signal adaptively for various situations and improve the measurement accuracy.

### SUMMARY

According to one embodiment, an electrophysiological signal measurement system is provided. The electrophysiological signal measurement system includes an electrode assembly, a variation adjustment device and a signal processing device. The electrode assembly is configured to receive an electrophysiological signal, a first electrical characteristic value and a second electrical characteristic value. The variation adjustment device includes a comparison unit and a searching unit. The comparison unit is configured to receive the first electrical characteristic value and the second electrical characteristic value, and determine whether a difference between the first electrical characteristic value and the second electrical characteristic value is greater than a threshold. The searching unit is configured to search for a plurality of amplitude calibration ratios corresponding to a plurality of frequencies when the difference between the first electrical characteristic value and the second electrical characteristic value is greater than the threshold. The signal processing device is configured to calibrate the electrophysiological signal according to the amplitude calibration ratios corresponding to the frequencies.

According to another embodiment, an electrophysiological signal adjustment method is provided. The electrophysiological signal adjustment method includes the following steps. An electrophysiological signal is received. A first electrical characteristic value and a second electrical characteristic value are received. Whether a difference between the first electrical characteristic value and the second electrical characteristic value is greater than a threshold is determined. A plurality of amplitude calibration ratios corresponding to a plurality of frequencies is searched when the difference between the first electrical characteristic value and the second electrical characteristic value is greater than the threshold. The electrophysiological signal is calibrated according to the amplitude calibration ratios corresponding to the frequencies.

According to an alternative embodiment, an electrode assembly is provided. The electrode assembly includes a first measuring electrode, a first ring electrode, a first surrounding electrode, a second measuring electrode, a second ring electrode and a second surrounding electrode. The first ring electrode surrounds the first measuring electrode. The first surrounding electrode surrounds the first ring electrode. The first measuring electrode and the second measuring electrode are configured to receive an electrophysiological signal. A location of the first measuring electrode is different from a location of the second measuring electrode. The second ring electrode surrounds the second measuring electrode. The second surrounding electrode surrounds the second ring electrode.

### BRIEF DESCRIPTION OF THE DRAWINGS

FIG. 1A shows an electrophysiological signal measurement system according to an embodiment.

FIG. 1B shows another usage mode of the electrophysiological signal measurement system.

FIG. 2 illustrates the relationship between the electrophysiological signal measurement system and the skin.

FIG. 3 shows a schematic diagram of the electrode assembly according to an embodiment.

FIG. 4 shows a block diagram of the electrophysiological signal measurement system according to an embodiment.

FIG. 5 shows a flowchart of the electrophysiological signal adjustment method according to an embodiment.

FIG. 6 shows a detailed flow chart of step S110 according to an embodiment.

FIG. 7 illustrates the calibration performed on one of the electrophysiological sub-signals.

FIG. 8 shows an electrophysiological signal measurement system according to another embodiment.

FIG. 9 shows a schematic diagram of the electrode assembly according to another embodiment.

FIG. 10 shows a block diagram of the electrophysiological signal measurement system according to another embodiment.

FIG. 11 shows a flow chart of an electrophysiological signal adjustment method according to another embodiment.

FIG. 12 shows a schematic diagram of the electrode assembly according to another embodiment.

FIG. 13 shows a block diagram of an electrophysiological signal measurement system according to another embodiment.

FIG. 14 shows a flow chart of an electrophysiological signal adjustment method according to another embodiment.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

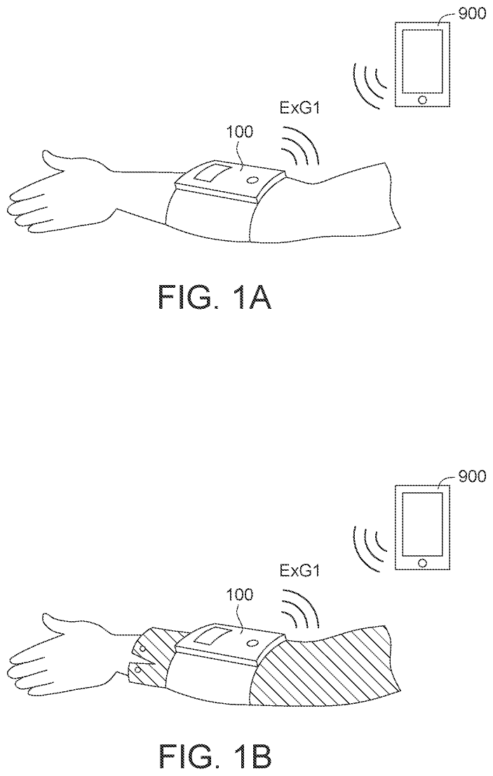

Please refer to FIG. 1A, which shows an electrophysiological signal measurement system 100 according to an embodiment. For example, the user wears the electrophysiological signal measurement system 100 on the arm to measure an electrophysiological signal ExG1. The electrophysiological signal ExG1 is, for example, an Electrocardiography signal (ECG signal), an Electromyography signal (EMG signal) or an Electroencephalography signal (EEG signal). The electrophysiological signal ExG1 can be transmitted to the mobile device 900 for the user to know its health status, or for further analysis and processing in the mobile device 900.

Please refer to FIG. 1B, which shows another usage mode of the electrophysiological signal measurement system 100. In another embodiment, the user can also use the electrophysiological signal measurement system 100 through clothing. The electrophysiological signal adjustment techniques proposed below are also suitable for this usage mode.

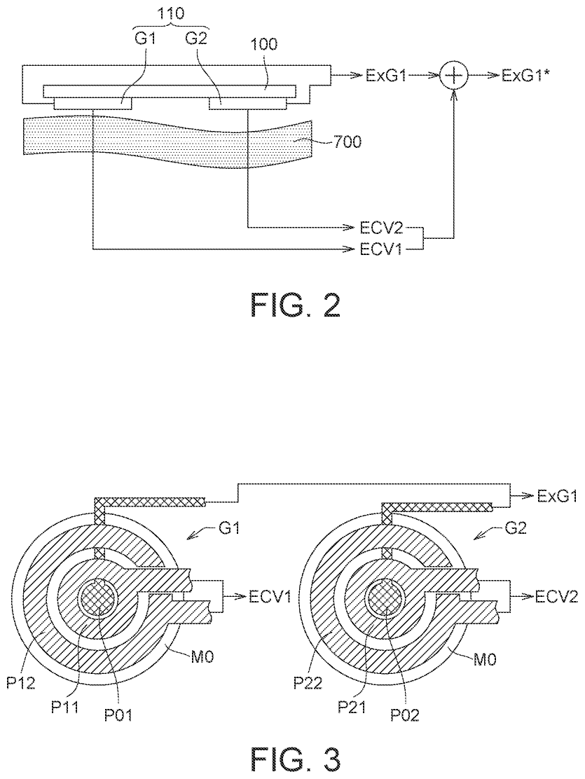

Please refer to FIG. 2, which illustrates the relationship between the electrophysiological signal measurement system 100 and the skin 700. An electrode assembly 110 of the electrophysiological signal measurement system 100 is in contact with or near the skin 700 for measurement. The electrode assembly 110 in FIG. 2 includes a first component G1 and a second component G2. The first component G1 and the second component G2 receive the electrophysiological signal ExG1. In addition to the electrophysiological signal ExG1, a first electrical characteristic value ECV1 is received via the first component G1, and a second electrical characteristic value ECV2 is received via the second component G2. The first electrical characteristic value ECV1 and the second electrical characteristic value ECV2 are, for example, capacitance values, or resistance values.

According to the first electrical characteristic value ECV1 and the second electrical characteristic value ECV2, the electrophysiological signal measurement system 100 can decide whether to calibrate the electrophysiological signal ExG1 to obtain a calibrated electrophysiological signal ExG1*.

Please refer to FIG. 3, which shows a schematic diagram of the electrode assembly 110 according to an embodiment. The electrode assembly 110 includes a first measuring electrode P01, a second measuring electrode P02, a first ring electrode P11, a first surrounding electrode P12, a second ring electrode P21, a second surrounding electrode P22 and an insulation material M0. The first measuring electrode P01, the first ring electrode P11 and the first surrounding electrode P12 form the first component G1. The second measuring electrode P02, the second ring electrode P21 and the second surrounding electrode P22 form the second component G2.

The first measuring electrode P01 and the second measuring electrode P02 are used to receive the electrophysiological signal ExG1. The first ring electrode P11 and the first surrounding electrode P12 are used to receive the first electrical characteristic value ECV1. The second ring electrode P21 and the second surrounding electrode P22 are used to receive the second electrical characteristic value ECV2. The location of the first measuring electrode P01 is different from the location of the second measuring electrode P02.

The first ring electrode P11 surrounds the first measuring electrode P01. The first surrounding electrode P12 surrounds the first ring electrode P11. The second ring electrode P21 surrounds the second measuring electrode P02. The second surrounding electrode P22 surrounds the second ring electrode P21. The insulation material M0 is disposed among the first measuring electrode P01, the second measuring electrode P02, the first ring electrode P11, the first surrounding electrode P12, the second ring electrode P21 and the second surrounding electrode P22. The first ring electrode P11 and the first surrounding electrode P12 are preferably arranged concentrically, and the shape is not limited, such as concentric circles, concentric rectangles, concentric polygons, etc. The areas of the first ring electrode P11 and the first surrounding electrode P12 are substantially equal. The second ring electrode P21 and the second surrounding electrode P22 are preferably arranged concentrically, and the shape is not limited, such as concentric circles, concentric rectangles, concentric polygons, etc. The areas of the second ring electrode P21 and the second surrounding electrode P22 are substantially equal. If the electrodes are arranged concentrically and the areas are similar, the impedance can be reduced and the accuracy of the measured capacitance value can be increased.

Through the design of the aforementioned electrode assembly 110, the first ring electrode P11 and the first surrounding electrode P12 can measure the first electrical characteristic value ECV1 at the location of the first measuring electrode P01. The second ring electrode P21 and the second surrounding electrode P22 can measure the second electrical characteristic value ECV2 at the location of the second measuring electrode P02. Once the relationship between the first electrical characteristic value ECV1 and the second electrical characteristic value ECV2 changes, it means that the electrophysiological signal ExG1 received by the first measuring electrode P01 and the second measuring electrode P02 also changes, so the electrophysiological signal measurement system 100 can determine whether the electrophysiological signal ExG1 needs to be calibrated to obtain the calibrated electrophysiological signal ExG1* (shown in FIG. 2) according to the first electrical characteristic value ECV1 and the second electrical characteristic value ECV2.

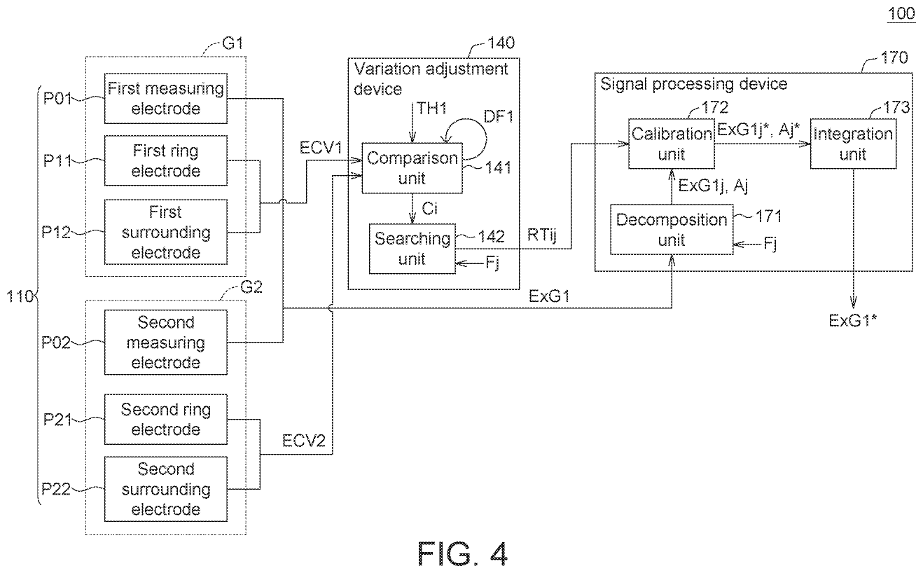

Please refer to FIG. 4, which shows a block diagram of the electrophysiological signal measurement system 100 according to an embodiment. The electrophysiological signal measurement system 100 includes the aforementioned electrode assembly 110, a variation adjustment device 140 and a signal processing device 170. The variation adjustment device 140 is used to determine amplitude calibration ratios RTij according to the first electrical characteristic value ECV1 and the second electrical characteristic value ECV2. The signal processing device 170 is used for calibrating the electrophysiological signal ExG1. The variation adjustment device 140 and/or the signal processing device 170 is, for example, a chip, a circuit, a circuit board, a computer program product, or a computer-readable recording medium.

The variation adjustment device 140 includes a comparison unit 141 and a searching unit 142. The signal processing device 170 includes a decomposition unit 171, a calibration unit 172 and an integration unit 173. The variation adjustment device 140 performs characteristic value comparison through the comparison unit 141, and performs data search through the searching unit 142. The signal processing device 170 performs signal decomposition through the decomposition unit 171, performs individual calibrations through the calibration unit 172, and finally performs integration through the integration unit 173 to obtain the final calibration result. The operation of the aforementioned components will be described in detail through the flow chart below.

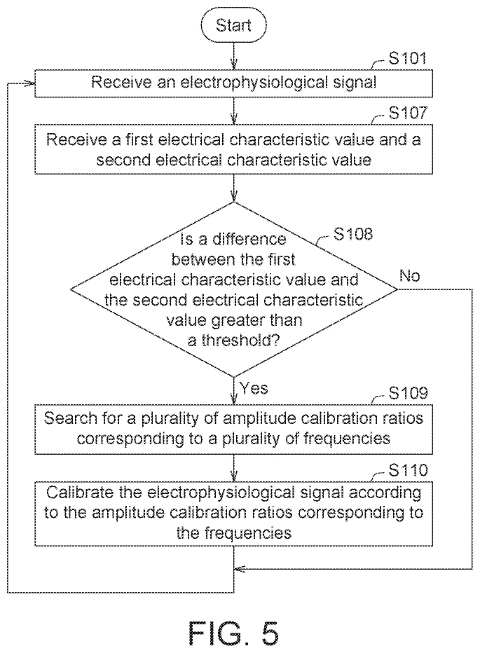

Please refer to FIG. 5, which shows a flowchart of the electrophysiological signal adjustment method according to an embodiment. In step S101, the first measuring electrode P01 and the second measuring electrode P02 of the electrode assembly 110 receive the electrophysiological signal ExG1. The electrophysiological signal ExG1 is, for example, the ECG signal or the EMG signal.

Next, in step S107, the first ring electrode P11 and the first surrounding electrode P12 of the electrode assembly 110 receive the first electrical characteristic value ECV1, and the second ring electrode P21 and the second surrounding electrode P22 of the electrode assembly 110 receives the second electrical characteristic value ECV2. The first electrical characteristic value ECV1 and the second electrical characteristic value ECV2 are, for example, capacitance values or resistance values.

Then, in step S108, the comparison unit 141 receives the first electrical characteristic value ECV1 and the second electrical characteristic value ECV2, and determines whether a difference DF1 between the first electrical characteristic value ECV1 and the second electrical characteristic value ECV2 is greater than a threshold TH1. If the difference DF1 between the first electrical characteristic value ECV1 and the second electrical characteristic value ECV2 is greater than the threshold TH1, then the process proceeds to step S109; if the difference DF1 between the first electrical characteristic value ECV1 and the second electrical characteristic value ECV2 is not greater than the threshold TH1, then the process proceeds to step S101. Generally speaking, when the user has a large dynamic action, it may cause that the first component G1 or the second component G2 of the electrode assembly 110 is not completely attached to the skin, and the first electrical characteristic value ECV1 and the second electrical characteristic value ECV2 may be inconsistent matched. Once the difference DF1 is greater than the threshold TH1, it is necessary to calibrate the electrophysiological signal ExG1, so the process proceeds to steps S109 to S110. The difference DF1 is, for example, the ratio between a differential signal and a common mode signal generated internally by the comparison unit 141 after receiving the first electrical characteristic value ECV1 and the second electrical characteristic value ECV2. The threshold TH1 is, for example, 0.02.

In the step S109, the searching unit 142 searches for the amplitude calibration ratios RTij corresponding to the frequencies Fj according to the first electrical characteristic value ECV1 and the second electrical characteristic value ECV2. In this step, the searching unit 142 performs searching, for example, according to an average value Ci of the first electrical characteristic value ECV1 and the second electrical characteristic value ECV2. For example, please referring to the following table I, when the average value Ci is “C1”, the amplitude calibration ratios RTij corresponding to the frequencies Fj which are “F1 to Fn” are “RT11 to RT1n.”

TABLE I

| average value Ci of the first | | |
|---|---|---|
| electrical characteristic | | |
| value ECV1 and the second | | amplitude |
| electrical characteristic | | calibration |
| value ECV2 | frequency Fj | ratio RTij |
| C1 | F1 | RT11 |
| | F2 | RT12 |
| | . . . | . . . |
| | Fn | RT1n |

Please referring to the following table two again, when the average value Ci is “C5”, the amplitude calibration ratios RTij corresponding to the frequencies Fj which are “F1 to Fn” are “RT51 to RT5n.”

TABLE II

| average value Ci of the first | | |
|---|---|---|
| electrical characteristic | | |
| value ECV1 and second | | amplitude |
| electrical characteristic | | calibration |
| value ECV2 | frequency Fj | ratio RTij |
| C5 | F1 | RT51 |
| | F2 | RT52 |
| | . . . | . . . |
| | Fn | RT5n |

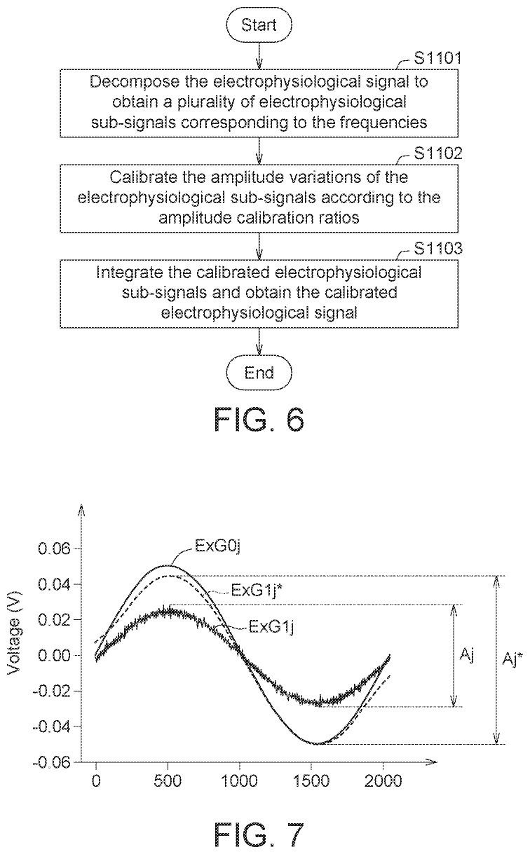

Afterwards, in step S110, the signal processing device 170 calibrates the electrophysiological signal ExG1 according to the amplitude calibration ratios RTij corresponding to the frequencies Fj. Please refer to FIG. 6, which shows a detailed flow chart of step S110 according to an embodiment. The step S110 includes steps S1101 to S1103.

In the step S1101, the composition unit 171 of the signal processing device 170 decomposes the electrophysiological signal ExG1 to obtain a plurality of electrophysiological sub-signals ExG1j corresponding to the frequencies Fj. Each of the electrophysiological sub-signals ExG1j has an amplitude variation Aj. Each of the amplitude variations Aj is, for example, the difference between the highest amplitude point and the lowest amplitude point, or, the difference between the center point of the AC wave signal and the highest amplitude point or the lowest amplitude point. As shown in Table III below, the electrophysiological signal ExG1 can be decomposed into a plurality of electrophysiological sub-signals ExG1j, such as “ExG11, ExG12, ExG1n”, whose corresponding frequencies Fj and corresponding amplitude variations Aj are “F1, F2, . . . , Fn” and “A1, A2, . . . , An” respectively.

TABLE III

| electrophysiological | | amplitude |
|---|---|---|
| sub-signal ExG1j | frequency Fj | variation Aj |
| ExG11 | F1 | A1 |
| ExG12 | F2 | A2 |
| . . . | . . . | . . . |
| ExG1n | Fn | An |

In one embodiment, the decomposition unit 171 decomposes the electrophysiological signal ExG1 by a signal decomposition algorithm. The signal decomposition algorithm is a combination of a Short time Fourier transform (STFT) and a Power Spectral Density Function (PSDF) algorithm, or the signal decomposition algorithm is a wavelet transform algorithm, or the signal decomposition algorithm is an Empirical Mode Decomposition (EMD) algorithm.

Then, in the step S1102, the calibration unit 172 calibrates the amplitude variations Aj of the electrophysiological sub-signals ExG1j according to the amplitude calibration ratios RTij corresponding to the frequencies Fj respectively. Please refer to FIG. 7, which illustrates the calibration performed on one of the electrophysiological sub-signals ExG1j. The amplitude variation Aj of this electrophysiological sub-signal ExG1j is only 24.43 mV. The calibration unit 172 calibrates the amplitude variation Aj of the electrophysiological sub-signal ExG1j to be the amplitude variation Aj* by an amplitude calibration ratio RTij, such as 51.1%. The calibrated amplitude variation Aj* is, for example, 50 mV. Comparing the calibrated electrophysiological sub-signal ExG1j* with the ideal electrophysiological sub-signal ideal electrophysiological signal ExG0j, the accuracy rate can reach 99.61%.

In one embodiment, for different electrophysiological sub-signals ExG1j, the amplitude calibration ratios RTij corresponding to the amplitude variations Aj may not be totally the same. The calibration unit 172 calibrates all of the electrophysiological sub-signals ExG1j.

Next, in step S1103, the integration unit 173 uses the inverse Fourier transform algorithm (frequency domain to time domain) to integrate the electrophysiological sub-signals ExG1j calibrated by the calibration unit 172 and obtains the calibrated electrophysiological signal ExG1*.

According to the above-described embodiment, after receiving the electrophysiological signal ExG1, the variation adjustment device 140 obtains the amplitude calibration ratios RTij. The signal processing device 170 can calibrate the electrophysiological signal ExG1 according to the amplitude calibration ratios RTij to obtain the calibrated electrophysiological signal ExG1*. The calibrated electrophysiological signal ExG1* overcomes the mismatch of electrical characteristic values, so that the electrophysiological signal measurement system 100 can obtain highly accurate measurement results even when the user has a large dynamic action.

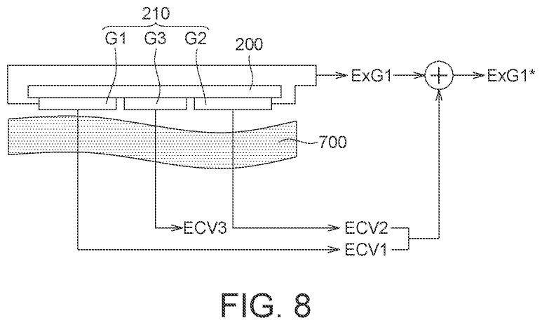

The aforementioned embodiment carries out the calibration of the electrophysiological signal ExG1 through the comparison of the first electrical characteristic value ECV1 and the second electrical characteristic value ECV2. In another embodiment, whether the electrical characteristic value has changed can be detected before the above comparison to save power. Please refer to FIG. 8, which shows an electrophysiological signal measurement system 200 according to another embodiment. An electrode assembly 210 in FIG. 8 includes the aforementioned first component G1, the aforementioned second component G2 and a third component G3. The first component G1 and the second component G2 receive the electrophysiological signal ExG1. The first component G1 receives the first electrical characteristic value ECV1, the second component G2 receives the second electrical characteristic value ECV2, and the third component G3 receives a third electrical characteristic value ECV3. The first electrical characteristic value ECV1, the second electrical characteristic value ECV2 and the third electrical characteristic value ECV3 are capacitance values, or resistance values.

Whether the first electrical characteristic value ECV1 and the second electrical characteristic value ECV2 need to be compared and whether the electrophysiological signal ExG1 needs to be calibrated are determined according to the third electrical characteristic value ECV3 to obtain the calibrated electrophysiological signal ExG1*.

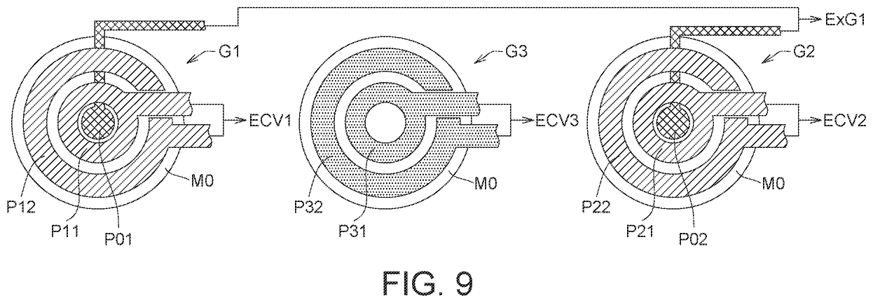

Please refer to FIG. 9, which shows a schematic diagram of the electrode assembly 210 according to another embodiment. The electrode assembly 210 includes the aforementioned first measuring electrode P01, the aforementioned second measuring electrode P02, the aforementioned first ring electrode P11, the aforementioned first surrounding electrode P12, aforementioned second ring electrode P21, the aforementioned second surrounding electrode P22, a third ring electrode P31, a third surrounding electrode P32 and the aforementioned insulation material M0.

The third ring electrode P31 is disposed between the first ring electrode P11 and the second ring electrode P21. The third surrounding electrode P32 surrounds the third ring electrode P31. The third ring electrode P31 and the third surrounding electrode P32 are used to receive the third electrical characteristic value ECV3. The insulation material M0 is disposed among the first measuring electrode P01, the second measuring electrode P02, the first ring electrode P11, the first surrounding electrode P12, the second ring electrode P21, the second surrounding electrode P22, the third ring electrode P31 and the third surrounding electrode P32. The third ring electrode P31 and the third surrounding electrode P32 are preferably arranged concentrically, and the shape is not limited, such as concentric circles, concentric rectangles, concentric polygons, etc. The areas of the third ring electrode P31 and the third surrounding electrode P32 are substantially equal. When the electrodes are arranged concentrically and the area is similar, the impedance can be reduced and the accuracy of the measured capacitance value can be increased.

Through the design of the aforementioned electrode assembly 210, the third ring electrode P31 and the third surrounding electrode P32 can measure the third electrical characteristic value ECV3 between the first measuring electrode P01 and the second measuring electrode P02. Once the third electrical characteristic value ECV3 changes greatly, it means that the first electrical characteristic value ECV1 and the second electrical characteristic value ECV2 may also change, so it can be determined whether the first electrical characteristic value ECV1 and the second electrical characteristic value ECV2 are needed to be compared according to the third electrical characteristic value ECV3.

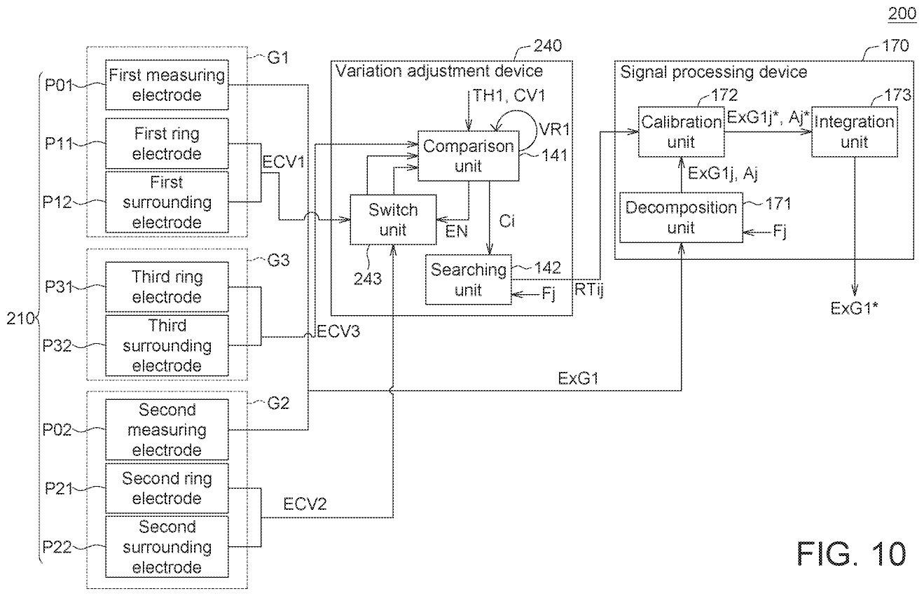

Please refer to FIG. 10, which shows a block diagram of the electrophysiological signal measurement system 200 according to another embodiment. The variation adjustment device 240 of the electrophysiological signal measurement system 200 further includes a switch unit 243. The comparison unit 141 of the variation adjustment device 240 can determine whether the switch unit 243 needs to be turned on according to the third electrical characteristic value ECV3, so that the comparison unit 141 receives the first electrical characteristic value ECV1 and the second electrical characteristic value ECV2 and compares them. The operation of each component is explained in detail through the flow chart below.

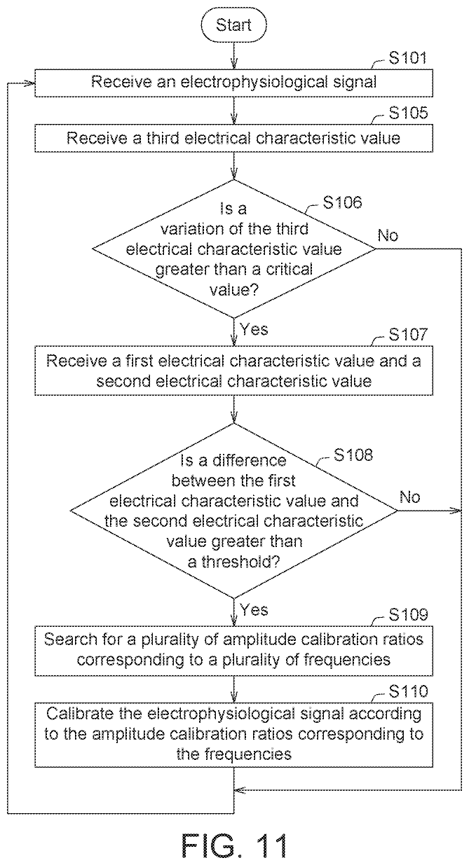

Please refer to FIG. 11, which shows a flow chart of an electrophysiological signal adjustment method according to another embodiment. The electrophysiological signal adjustment method of this embodiment further includes steps S105 to S106. In the step S105, the third ring electrode P31 and the third surrounding electrode P32 of the electrode assembly 210 receive the third electrical characteristic value ECV3. In the step S106, the comparison unit 141 determines whether a variation VR1 of the third electrical characteristic value ECV3 is greater than a critical value CV1. The critical value CV1 is, for example, 33%. If the variation VR1 of the third electrical characteristic value ECV3 is greater than the critical value CV1, then the process proceeds to the step S107; if the variation VR1 of the third electrical characteristic value ECV3 is not greater than the critical value CV1, then the process returns to the step S101. In this step, the variation VR1 of the third electrical characteristic value ECV3 is the variation with time. Once the third electrical characteristic value ECV3 changes drastically, it is necessary to compare the first electrical characteristic value ECV1 and the second electrical characteristic value ECV2, so the process proceeds to the steps S107 to S108.

As shown in FIG. 10, when the comparison unit 141 determines that the variation VR1 of the third electrical characteristic value ECV3 is greater than the critical value CV1, it will output an enable signal EN to the switch unit 243, so that the comparison unit 141 receives the first electrical characteristic value ECV1 and the second electrical characteristic value ECV2 in the step S107 and performs the determination in the step S108.

According to the aforementioned embodiment, only when the third electrical characteristic value ECV3 has a large change, the comparison unit 141 performs the comparison between the first electrical characteristic value ECV1 and the second electrical characteristic value ECV2, so as to save power consumption.

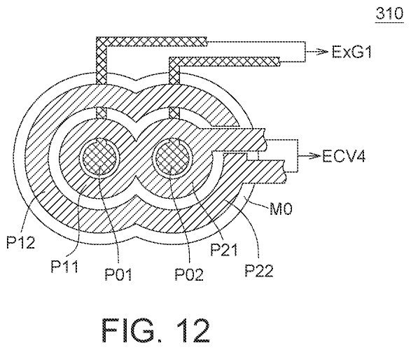

In addition, when the muscle group to be measured or the area is small, the aforementioned electrode assemblies 110, 210 can be integrated. Please refer to FIG. 12, which shows a schematic diagram of the electrode assembly 310 according to another embodiment. In the embodiment of FIG. 12, the first ring electrode P11 of the electrode assembly 310 is connected to the second ring electrode P21. The first surrounding electrode P12 of electrode assembly 310 is connected to the second surrounding electrode P22. In this embodiment, the first ring electrode P11, the second ring electrode P21, the first surrounding electrode P12 and the second surrounding electrode P22 receive a fourth electrical characteristic value ECV4. According to the situation of the fourth electrical characteristic value ECV4 (such as variation), it can be decided whether to calibrate the electrophysiological signal ExG1.

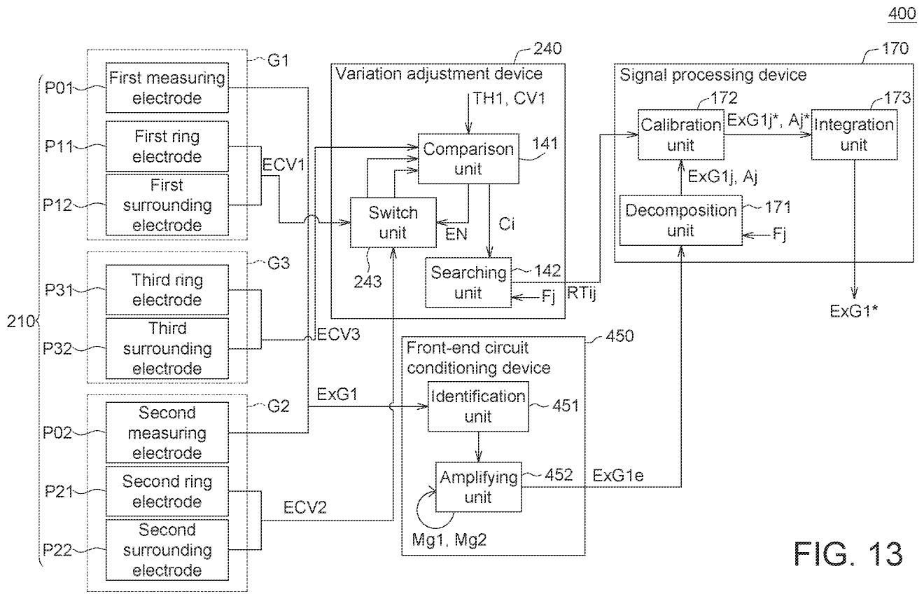

Furthermore, for the subject with muscle injury or the elderly, the amplitude of the electrophysiological signal ExG1 will be weak, and it is difficult to measure and interpret. Please refer to FIG. 13, which shows a block diagram of an electrophysiological signal measurement system 400 according to another embodiment. A front-end circuit conditioning device 450 includes an identification unit 451 and an amplifying unit 452. The identification unit 451 is used to identify the signal type. The amplifying unit 452 is used for signal amplification. The front-end circuit conditioning device 450 is, for example, a chip, a circuit, a circuit board, a computer program product or a computer-readable recording medium. The electrophysiological signal measurement system 400 can perform adaptive signal amplification through the front-end circuit conditioning device 450 to compensate the weak electrophysiological signal ExG1. The operation of each component is described in detail with the flow chart below.

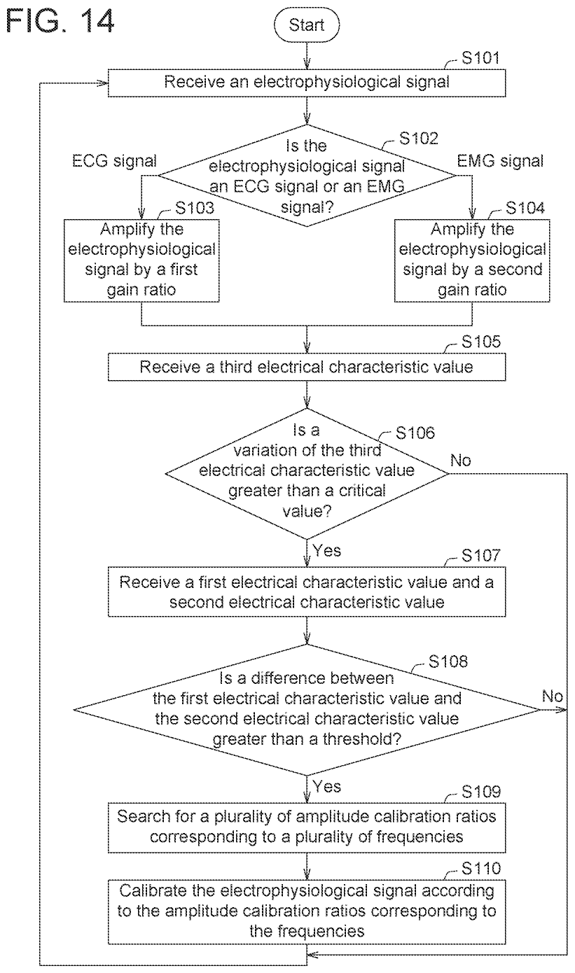

Please refer to FIG. 14, which shows a flow chart of an electrophysiological signal adjustment method according to another embodiment. The electrophysiological signal adjustment method of this embodiment further includes steps S102 to S104 to perform adaptive signal amplification. In step S102, the identification unit 451 of the front-end circuit conditioning device 450 identifies that the electrophysiological signal ExG1 is the ECG signal or the EMG signal. If the electrophysiological signal ExG1 is the ECG signal, then the process proceeds to the step S103; if the electrophysiological signal ExG1 is the EMG signal, then the process proceeds to the step S104. For example, if the electrophysiological signal ExG1 is less than or equal to 3 mV, the identification unit 451 deems that the electrophysiological signal ExG1 is the ECG signal. If the electrophysiological signal ExG1 is greater than 3 mV and less than or equal to 5 mV, the identification unit 451 deems that the electrophysiological signal ExG1 is the EMG signal.

In step S103, the amplifying unit 452 of the front-end circuit conditioning device 450 amplifies the electrophysiological signal ExG1 by a first gain ratio Mg1. The first gain ratio Mg1 is, for example, 2 times.

In step S104, the amplifying unit 452 of the front-end circuit conditioning device 450 amplifies the electrophysiological signal ExG1 by a second gain ratio Mg2. The second gain ratio Mg2 is, for example, 3 times.

The amplified electrophysiological signal ExG1e is inputted to the signal processing device 170 for performing the calibration in the steps S105 to S110.

According to the above-mentioned embodiment, when the user has a large dynamic action, and the electrode assembly 110 is not completely attached to the skin, the calibration of the electrophysiological signal ExG1 can be performed. Furthermore, for subjects with muscle injury, the electrophysiological signal ExG1 can also compensate appropriately. Therefore, the electrophysiological signal ExG1 can be adjusted adaptively for various situations, which greatly improves the measurement accuracy.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. An electrophysiological signal measurement system, comprising:
an electrode assembly, configured to receive an electrophysiological signal, a first electrical characteristic value and a second electrical characteristic value;
a variation adjustment device, including:
a comparison unit, configured to receive the first electrical characteristic value and the second electrical characteristic value, and determine whether a difference between the first electrical characteristic value and the second electrical characteristic value is greater than a threshold; and
a searching unit, configured to search for a plurality of amplitude calibration ratios corresponding to a plurality of frequencies when the difference between the first electrical characteristic value and the second electrical characteristic value is greater than the threshold; and
a signal processing device, configured to calibrate the electrophysiological signal according to the amplitude calibration ratios corresponding to the frequencies.

2. The electrophysiological signal measurement system according to claim 1, wherein the electrode assembly includes:
a first measuring electrode;
a first ring electrode, surrounding the first measuring electrode;
a first surrounding electrode, surrounding the first ring electrode, wherein the first ring electrode and the first surrounding electrode are configured to receive the first electrical characteristic value;
a second measuring electrode, wherein the first measuring electrode and the second measuring electrode are configured to receive the electrophysiological signal, and a location of the first measuring electrode is different from a location of the second measuring electrode;
a second ring electrode, surrounding the second measuring electrode;
a second surrounding electrode, surrounding the second ring electrode, wherein the second ring electrode and the second surrounding electrode are configured to receive the second electrical characteristic value.

3. The electrophysiological signal measurement system according to claim 2, wherein the first ring electrode and the first surrounding electrode are arranged concentrically, an area of the first ring electrode and an area of the first surrounding electrode are substantially equal, the second ring electrode and the second surrounding electrode are arranged concentrically, and an area of the second ring electrode and an area of the second surrounding electrode are substantially equal.

4. The electrophysiological signal measurement system according to claim 2, wherein the electrode assembly further includes:
a third ring electrode, disposed between the first ring electrode and the second ring electrode;
a third surrounding electrode, surrounding the third ring electrode, wherein the third ring electrode and the third surrounding electrode are configured to receive a third electrical characteristic value.

5. The electrophysiological signal measurement system according to claim 4, wherein the third ring electrode and the third surrounding electrode are arranged concentrically, and an area of the third ring electrode and an area of the third surrounding electrode are equal.

6. The electrophysiological signal measurement system according to claim 4, wherein the comparison unit is further configured to determine whether a variation of the third electrical characteristic value is greater than a critical value, the comparison unit receives the first electrical characteristic value and the second electrical characteristic value only when the variation of the third electrical characteristic value is greater than the critical value.

7. The electrophysiological signal measurement system according to claim 1, wherein the first electrical characteristic value and the second electrical characteristic value are capacitance values, or resistance values.

8. The electrophysiological signal measurement system according to claim 1, further comprising:
a front-end circuit conditioning device, including:
an identification unit, configured to identify whether the electrophysiological signal is an Electrocardiography signal (ECG signal) or an Electromyography signal (EMG signal); and
an amplifying unit, wherein if the electrophysiological signal is the ECG signal, the amplifying unit amplifies the electrophysiological signal by a first gain ratio; if the electrophysiological signal is the EMG signal, the amplifying unit amplifies the electrophysiological signal by a second gain ratio which is larger than the first gain ratio.

9. The electrophysiological signal measurement system according to claim 8, wherein if the electrophysiological signal is less than or equal to 3 mV, then the identification unit deems that the electrophysiological signal is the ECG signal; if the electrophysiological signal is larger than 3 mV and less than or equal to 5 mV, then the identification unit deems the electrophysiological signal is the EMG signal.

10. An electrophysiological signal adjustment method, comprising:
receiving an electrophysiological signal;
receiving a first electrical characteristic value and a second electrical characteristic value;
determining whether a difference between the first electrical characteristic value and the second electrical characteristic value is greater than a threshold;
searching for a plurality of amplitude calibration ratios corresponding to a plurality of frequencies when the difference between the first electrical characteristic value and the second electrical characteristic value is greater than the threshold; and
calibrating the electrophysiological signal according to the amplitude calibration ratios corresponding to the frequencies.

11. The electrophysiological signal adjustment method according to claim 10, wherein a location for measuring the first electrical characteristic value is different from a location for measuring the second electrical characteristic value.

12. The electrophysiological signal adjustment method according to claim 11, further comprising:
receiving a third electrical characteristic value; and
determining whether a variation of the third electrical characteristic value is greater than a critical value;
wherein the step of receiving the first electrical characteristic value and the second electrical characteristic value is performed only when the variation of the third electrical characteristic value is greater than the critical value.

13. The electrophysiological signal adjustment method according to claim 10, wherein the first electrical characteristic value and the second electrical characteristic value are capacitance values, or resistance values.

14. The electrophysiological signal adjustment method according to claim 10, further comprising:
identifying whether the electrophysiological signal is an Electrocardiography signal (ECG signal) or an Electromyography signal (EMG signal);
amplifying the electrophysiological signal by a first gain ratio, if the electrophysiological signal is the ECG signal; and
amplifying the electrophysiological signal by a second gain ratio which is larger than the first gain ratio, if the electrophysiological signal is the EMG signal.

15. The electrophysiological signal adjustment method according to claim 14, wherein if the electrophysiological signal is less than or equal to 3 mV, then the electrophysiological signal is the ECG signal; if the electrophysiological signal is larger than 3 mV and less than or equal to 5 mV, then the electrophysiological signal is the EMG signal.

16. An electrode assembly, comprising:
a first measuring electrode;
a first ring electrode, surrounding the first measuring electrode;
a first surrounding electrode, surrounding the first ring electrode;
a second measuring electrode, wherein the first measuring electrode and the second measuring electrode are configured to receive an electrophysiological signal, a location of the first measuring electrode is different from a location of the second measuring electrode;
a second ring electrode, surrounding the second measuring electrode; and
a second surrounding electrode, surrounding the second ring electrode.

17. The electrode assembly according to claim 16, wherein the first ring electrode and the first surrounding electrode are arranged concentrically, an area of the first ring electrode and an area of the first surrounding electrode are substantially equal, the second ring electrode and the second surrounding electrode are arranged concentrically, and an area of the second ring electrode and an area of the second surrounding electrode are substantially equal.

18. The electrode assembly according to claim 17, further comprising:
a third ring electrode, disposed between the first ring electrode and the second ring electrode; and
a third surrounding electrode, surrounding the third ring electrode.

19. The electrode assembly according to claim 18, wherein the third ring electrode and the third surrounding electrode are arranged concentrically, and an area of the third ring electrode and an area of the third surrounding electrode are equal.

20. The electrode assembly according to claim 16, wherein the first ring electrode is connected to the second ring electrode, and the first surrounding electrode is connected to the second surrounding electrode.
